(19) United States
(12) Patent Application Publication
(10) Pub. No.: US 2019/0014980 A1
(43) Pub. Date: Jan. 17, 2019

(54) APPARATUS AND METHOD FOR FLEXIBLE BOUGIE AND STYLET FOR DIFFICULT INTUBATIONS

(71) Applicant: Joshua J Herskovic, Highland Park, IL (US)

(72) Inventor: Joshua J Herskovic, Highland Park, IL (US)

(21) Appl. No.: 15/647,546

(22) Filed: Jul. 12, 2017

(51) Int. Cl. A61B 1/267; A61B 1/00; A61M 16/04

(52) U.S. Cl. CPC A61B 1/267; A61B 1/05; A61M 16/0488; A61B 1/00052

(57) ABSTRACT

A stylet for providing access to an airway of a patient for insertion of an endotracheal tube for intubation is presented. The stylet is a hollow tube with a proximal and a distal end. The proximal end of the stylet has an actuator for, among other things, releasing the stylet when the ETT has been properly situated in the trachea of a patient. The stylet and bougie, if used, is removed prior to the balloon of the ETT being inflated to keep the ETT in the trachea. The stylet also is shaped prior to the insertion into the mouth and throat of the patient. The preformed shape of the stylet is consistent with the pathway to the trachea.

### BACKGROUND

### 1. Field of the Invention

The present invention relates to improvements in devices designed to facilitate an intubation procedure, such as placement of an endotracheal tube.

### 2. Background

Endotracheal intubation is a core technique in anesthesia (and critical care medicine). It is the gold standard method to provide oxygenation and ventilation to a patient under general anesthesia. It requires the passage of an endotracheal tube (ETT) through the vocal cords into the trachea.

The primary and traditional method of endotracheal intubation involves direct laryngoscopy with a laryngoscope. The laryngoscope has a gently curved blade which is passed into the mouth and sits in the oropharynx to provide a direct line of sight to the vocal cords. Most patients can be successfully intubated using this technique; however, circumstances where this is difficult or impossible occur relatively regularly in anesthesia and other areas of critical care.

Difficult intubation has the potential to result in great patient harm from inadequate oxygenation causing death, brain damage and/or heart attacks. Difficult intubation requiring multiple or repeated attempts can also result in trauma to the airway. Management of these scenarios has been and continues to be a major focus in anesthesia and critical care medicine. This has resulted in multiple “airway” management strategies and the development of improved equipment.

Although there can be a variety of causes that result in a difficult intubation, ultimately there are two main factors that are responsible:

Difficulty in visualizing the vocal cords and/or

Difficulty in directing an ETT through the vocal cords into the airway.

These two factors are generally managed by using a video laryngoscope (to improve visualization of the vocal cords) and/or a specialized ETT introducer (to guide the ETT through the vocal cords). However, both of these techniques have limitations which can still result in a difficult or failed intubation for various reasons.

For difficulty in directing the ETT, successful intubation requires the ETT to travel along the pathway between the mouth and vocal cords into the trachea.

Referring to FIG. 1, good visualization of the vocal cords 110 of a patient 100 during traditional direct laryngoscopy generally implies that there is a shallow (gently curved) pathway for the ETT to follow within the oropharynx and that intubation will be easy. This however may not be the case and despite good visualization, the ETT cannot be directed through the vocal cords 110.

Poor visualization of the vocal cords 110 during direct laryngoscopy often means that there is a more sharply angled pathway that the ETT must follow in the oropharynx. Video laryngoscopy 120 provides a better view of the vocal cords 110 in this case. However, because of physical factors associated with the video laryngoscopy 120, this angle may become acute with less space in the posterior portion of the oropharynx to work with. This sharp angle of approach can make it nearly impossible to direct an ETT through the vocal cords 110 without using a specialized introducer 130.

The introducers 130 have limitations and can fail. When an introducer 130 has to follow a very sharp angle of approach in the oropharynx, it can be difficult to direct the tip 140 of the introducer 130 through the vocal cords 110 as shown in FIG. 2 and FIG. 3. The introducer 130 can be bent into shape of the angle of approach in the oropharynx but two force factors come into play when determining proper alignment and direction into the vocal cords 110. Force is applied along the vector of the shaft which must then be transferred to align the tip 140 of the introducer 130 towards the vocal cords 110. Even if the introducer 130 successfully traverses the oropharynx into the vocal cords 110, it may become stuck in the wall of the trachea as shown in FIG. 4, which can make it difficult to direct the ETT 510, shown in FIG. 5, into the trachea despite having the introducer 130 successfully traverse into the vocal cords 110 (See FIG. 4). Thus in FIG. 5, the inflation of the balloon 520 of the ETT 510 does not secure the ETT in the trachea of the patient 100.

A first type of special introducer, e.g. a stylet, can be used to provide a guide for the ETT such that it is directed thru the vocal cords and into the trachea. This works often for difficult intubations but is not always successful. If the stylet is too flexible or is too rigid, the stylet may not be able to successfully navigate the vocal cords or provide a viable pathway for the ETT. Because of the sharp angles of the oropharynx as described above, a rigid introducer is beneficial for video laryngoscopy because a malleable introducer will lose it curvature by the time it navigates thru the oropharynx. However, because of limited space in the oropharynx, the rigid introducer with cannot be constructed long enough to successfully navigate the vocal cords or provide a viable pathway for the ETT.

A second type of special introducer, e.g. a bougie, can be used but currently requires the removal and disassembly of the ETT with the stylet or alternatively requires the anesthesiologist to setup another ETT for the bougie attempt. The special introducer is then inserted into the mouth and positioned in or near the trachea, only then can the ETT be guided over the inserted specialized introducer. This can be costly in the short period as the anesthesiologist has to provide oxygenation and ventilation for the patient. From the time the patient goes to sleep until the ETT is secured in the trachea, the patient is without oxygenation and ventilation. Therefore, the anesthesiologist is hesitant to lose time by disassembling the ETT with stylet setup or alternatively setup another ETT for a bougie attempt.

Accordingly, there exists a need to provide an improved device less prone to problems such as those described above. The present invention seeks to lessen these problems by providing a device which allows the intubation of a patient without significant difficulties associated with conventional devices.

### BRIEF DESCRIPTION OF THE DRAWINGS

FIG. 1 is a partial cross-sectional side view of a conventional introducer being used with a standard laryngoscope.

FIG. 2 is a partial cross-sectional side view of an attempt to direct a tip of a conventional introducer through the vocal cords using a standard laryngoscope.

FIG. 3 is a partial cross-sectional side view of the further progression of the attempt of FIG. 2.

FIG. 4 is a second partial cross-sectional side view of the further progression of the attempt of FIG. 3.

FIG. 5 is a partial cross-sectional side view showing a failed securing of the ETT in furtherance of the attempt of FIG. 4.

FIG. 6 is a view of a stylet in accordance with the preferred embodiment.

FIG. 7 is a side view of the stylet of FIG. 6.

FIG. 8A and FIG. 8B are a close-up view of the opening in the handle of the stylet shown in FIG. 6.

FIG. 9 is a view of the bougie in accordance with the preferred embodiment.

FIG. 10 is a view of a bougie with multiple slot patterns.

FIG. 11 is a close-up view of the proximal portion of the bougie of FIG. 9.

FIG. 12 is a close-up view of the distal portion of the bougie of FIG. 10.

FIG. 13 is a view of a bougie and a stylet prior to combining of the two.

FIG. 14 is a view of the bougie entered into the stylet being of FIG. 13.

FIG. 15 is a view of the bougie being rotated in the stylet of FIG. 14.

FIG. 16 is a view of a pre-packaged bougie and stylet combination.

FIG. 17 is a further view of the progress of the bougie progress in the combination of FIG. 16.

FIG. 18 is yet another view of the progress of the bougie progress in the combination of FIG. 17

FIG. 19 is a view of the stylet with a flexible tip.

FIG. 20 is a cross-view of the bougie of FIG. 19.

FIG. 21A and FIG. 21B is a flowchart representing the process of intubation.

### DETAILED DESCRIPTION

Reference will now be made in detail to the present preferred embodiments, examples of which are illustrated in the accompanying drawings. All terms in the plural shall also be taken as singular and vice-versa. Further, any reference to he shall also be applicable to she and vice-versa.

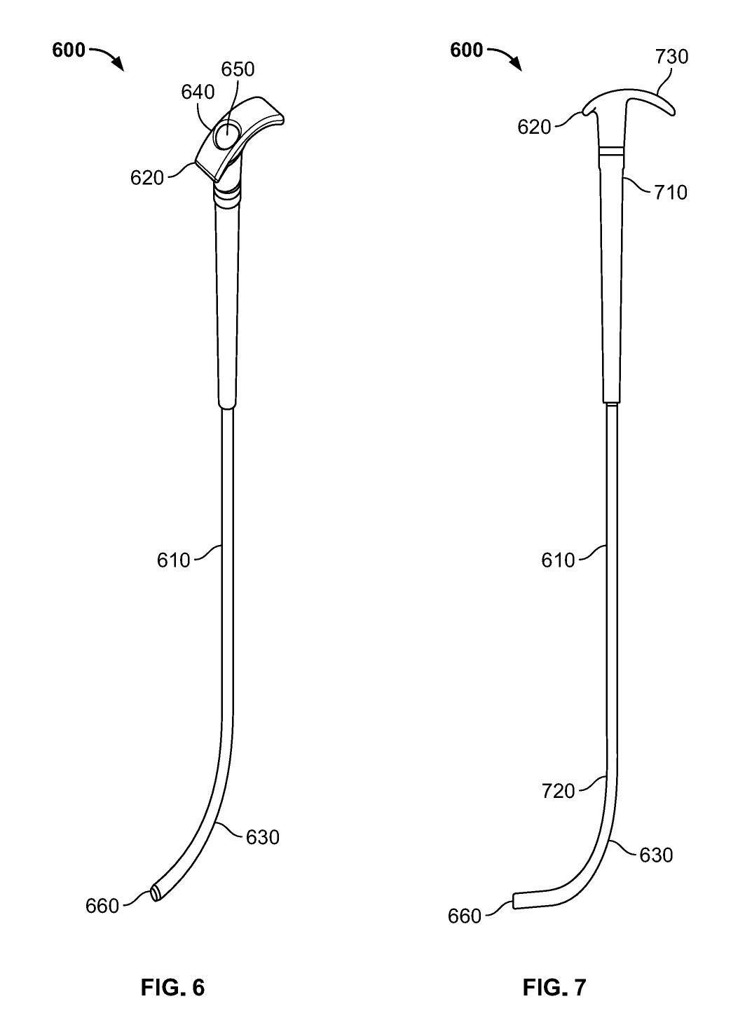

Referring to FIG. 6, an improved introducer, preferably a stylet, is indicated generally by the reference numeral 600. The stylet 600, designed for single use only, having a substantially tubular element 610. Fabricated of a hollow, semi-rigid, medical grade material which is generally circular in transverse cross-sections, the tubular element 610 preferably measures, at most, approximately 6.0 mm in diameter in order to ease its insertion, while encased in the tube 40, between the vocal cords. The medical grade material may be any material that may be used to manufacture a medical device such as plastic, metal, etc. The tubular element 610 has an actuator 620 and a curved section 630.

The actuator 620 has a solid portion 640 and an opening 650. The actuator 620 may be a handle, knob, dial, etc. The solid portion 640 may have any shape such as tapered, square, oblique, etc. The solid portion 640 is made to be easily controlled by a medical provider. The solid portion 640 may be used to release the stylet 600 once the ETT is secured in the trachea.

The curved section 630 is molded to a common curvature of the path towards the trachea. This allows for the stylet 600 to easily glide past the vocal cords by having a tip 660 that follow the contour of the path to the trachea such that the ETT may be placed properly to navigate the vocal cords and be placed in the trachea.

Referring to FIG. 7, a side view of the stylet 600 of FIG. 6 is shown. The stylet 600 has a tubular element 610 has a proximal end 710 and a distal end 720. The stylet 600 has an actuator 620 at the proximal end 710 and a curved section 630 before the tip 660 at the distal end 720. At the proximal end 710, the actuator 620 is a handle with a tapered portion 730. The actuator 620 performs multiple duties such as a handle for controlling the stylet 600, ease of release of the stylet 600 once the ETT, not shown, is securely position in the trachea, etc. The tapered portion 730 of the stylet 600 joins the actuator 620 to the tubular element 610. The tapering of the actuator 620 allows the ETT to position correctly on the stylet 600.

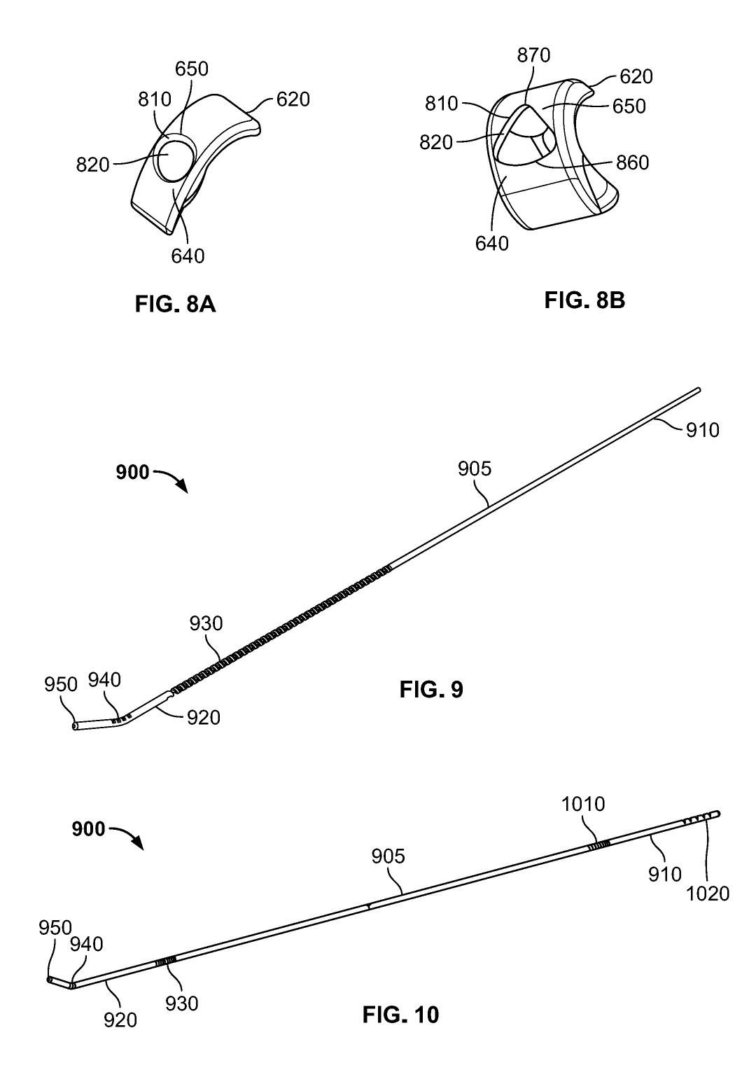

FIG. 8A and FIG. 8B show views of the opening 650 as shown in FIG. 7. Referring to FIG. 8A, the opening 650 has an outside edge 810 and an inside edge 820. The opening 650 is located over the hollow portion of the stylet 600 providing a medical provider the ability to position a second introducer into the stylet 600. Thus, the stylet 600 becomes a host for a second introducer without having to disconnect any other devices, such as the ETT. The opening 650 of the actuator 620 may be any shape, the shape shown in FIG. 8A is substantially round in shape and centered in the middle of the actuator 620. FIG. 8B shows a shape of the opening 650 of the actuator 620 to be non-round and off-center of the actuator 620. One of ordinary skill in the art may determine other locations, offsets and shapes of the opening, which are all contained within the scope of this application.

Referring to FIG. 8A, the outside edge 810 of the opening 650 of the actuator 620 is at least as the same size as the inside edge 840 of the opening 650 of the actuator 620. The inside edge 820 of the opening 650 of the actuator 620 may be smaller than the outside edge 810 of the opening 650 the actuator 620 to allow for any second introducer to be easily position in the stylet 600 and the second introducer may also secure in the stylet 600. Further the distance between the outside edge 810 of the opening 620 and the inside edge 820 of the opening 620 may vary to allow the second introducer to be positioned at an angle for ease of the medical provider as shown in FIG. 8B. Further, the distance between the inside edge 820 of the opening 620 and the outside edge 810 of the opening 620 may vary to allow for easier insertion of the second introducer, as shown in FIG. 8B.

Referring now to FIG. 9, an improved introducer, preferably a bougie, is indicated generally by the reference numeral 900. The bougie 900 can be designed for single use or for multiple use, has a substantially tubular element 905. Fabricated of a flexible, medical grade material which is generally circular in transverse cross-sections, the bougie 900 preferably measures, at most, approximately 6.0 mm in diameter in order to ease its insertion, while encased in the tubular element 605 of the stylet 600 of FIG. 6. The medical grade material may be any material that may be used to manufacture a medical device such as plastic, metal, etc.

The bougie 900 has a distal portion 920 and a proximal portion 910. The distal portion 920 of the bougie 900 is generally curved after the slotted pattern 930 and prior to the tip 950 of the bougie 900. The slotted pattern 930 may be a straight pattern, a corkscrew, etc. The curve of the distal portion 920 of the bougie 900 has a curve pattern 940 used to allow the bougie 900 to bend easily to accommodate the different paths associated with the airway of a patient. FIG. 9 shows that the curve pattern 940 of the distal portion 920 of the bougie 900 is a plurality of spaced apart, transverse slots so positioned that the midsection of each slot is disposed on only one side of the bougie's longitudinal centerline but the slots of the curve pattern 940 may be continual around the curve of the distal portion 920.

The slotted pattern 930 located on the distal portion 920 of the bougie 900 may be one or more slotted patterns 930, 1010, as shown in FIG. 10 where a second slotted pattern 1010 is located in the proximal portion 910 of the bougie 900. The patterns may be of any pattern, such as corkscrew, diagonal, etc. Each of the one or more slotted patterns 930, 960 may be identical to another of the slotted patterns 930, 1010 or may be different. The length of the one or more slotted patterns 930, 1010 may be of the same length or may be different lengths. Each of the one or more slotted patterns 930, 1010 is a continual slot circulating around the shaft of the introducer. The proximal end 910 of the bougie 900 also has a tip 1020. The tip may be a flexible tip, coude tip, or any type of tip.

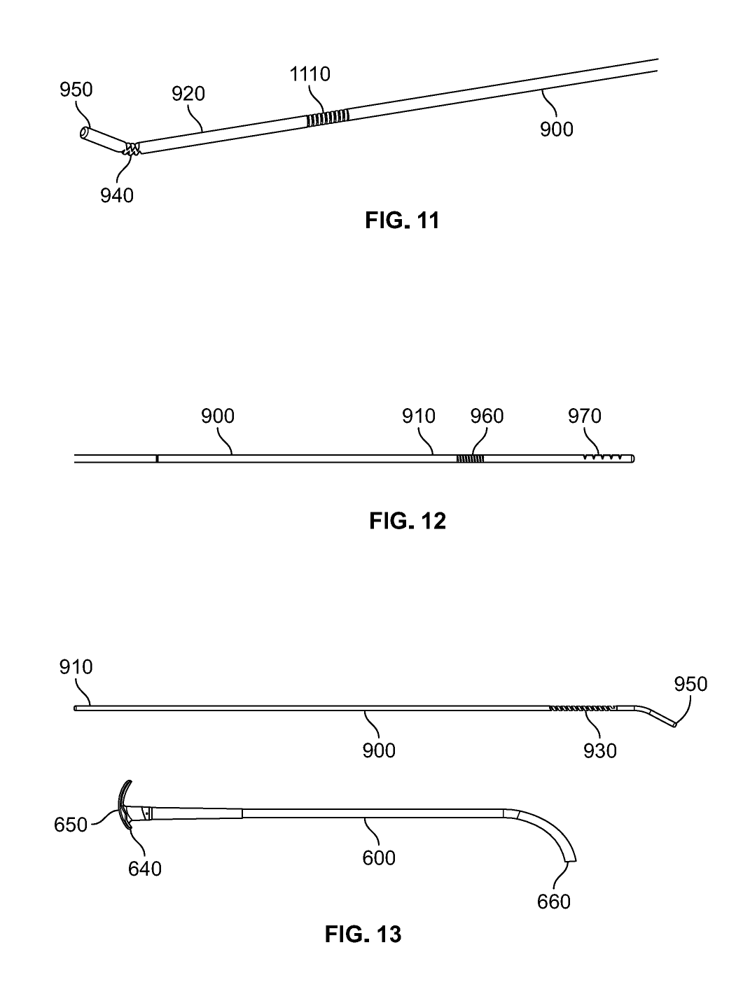

FIG. 11 and FIG. 12 show close up views of the distal portion 910 and the proximal portion 920 of the bougie 900 as well as the slotted patterns 930 and 960. Referring now to FIG. 11, the slotted pattern 1110 of the bougie 900 is show as having a different pattern to the slotted pattern 930 of FIG. 9 and also has a length that is shorter than the slotted pattern 930 of FIG. 9.

Referring now to FIG. 13, the bougie 900 of FIG. 9 and the Stylet 600 of FIG. 6 are shown. The stylet 600 has a handle 640 that has an opening 650. The opening 650 of the stylet 600 can be any shape, two examples are shown in FIG. 8A and FIG. 8B. The opening 650 of the stylet 600 is of such size as to accommodate the bougie 900.

Prior to use, the stylet 600 with the actuator 640 is inserted into an endotracheal tube, not shown. During use of this combination ETT/stylet assembly, the stylet 600 can be employed to maneuver the tip 660 of the stylet 600 past a patient's tongue and into the patient's vocal cords. After the tip 660 of the stylet 600 has been inserted, the ETT 510 is inserted further into the patient's airway. Once the assembly is properly placed anteriorly into the trachea and the stylet 600 is withdrawn. The ETT's 510 balloon 520 is inflated, locking the ETT 510 in place, allowing the patient to breathe/be ventilated.

The stylet 600 is hollow and has a tip 660 which allows for the tip 950 of the bougie 900 to extend beyond the tip 660 of the stylet 600. The proximal portion 910 of the bougie 900 is used to push and guide the bougie 900 thru the vocal cords and into the trachea. The slotted pattern 930 of the bougie 900 allows for the movement and torque exerted on the proximal portion 910 to be passed to the tip 950 of the bougie 900. Once the bougie 900 has passed the vocal cords and entered the trachea, the ETT 510 can then be slid down the extended bougie tip 950 into the trachea. Once the ETT 510 is properly placed anteriorly into the trachea, the ETT's 510 balloon 520 is inflated, locking the ETT 510 in place; and the stylet 600 and the bougie 900 are withdrawn by pulling up on the actuator 640, and the balloon 520 of the ETT 510 is inflated thereby locking the ETT 510 in place allowing the patient 100 to breathe/be ventilated.

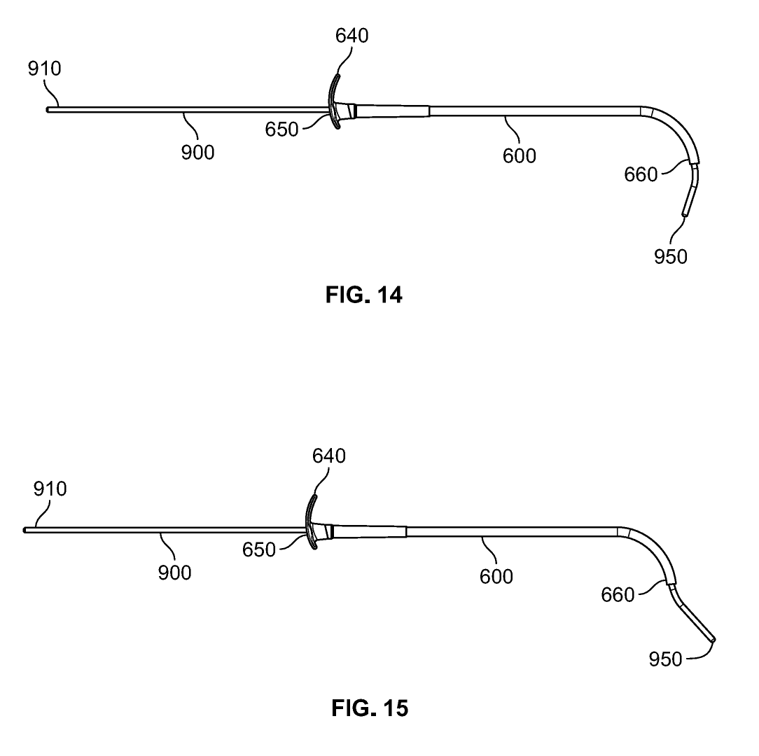

Moving now to FIG. 14 and FIG. 15, the progress the bougie being inserted into the stylet 600 is shown. FIG. 14, show that the bougie 900 has been inserted into the stylet 600 at the opening 650 of the actuator 640. The tip 950 of the bougie 900 can be seen just passing out of the tip 660 of the stylet 600. FIG. 15, shows that torque has been applied to the bougie 900 at the proximal end 910, thus translocating smooth rotational motion to the tip 950 of the bougie 900 as shown in FIG. 15, this can be performed with the stylet 600 or stand-alone.

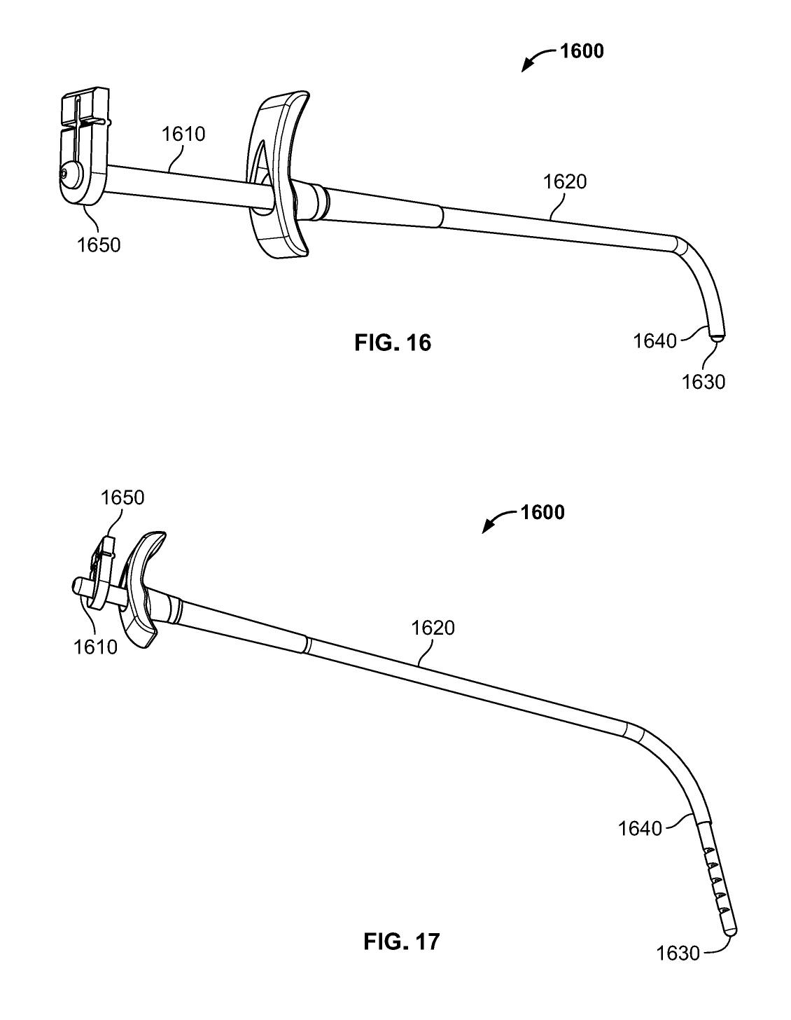

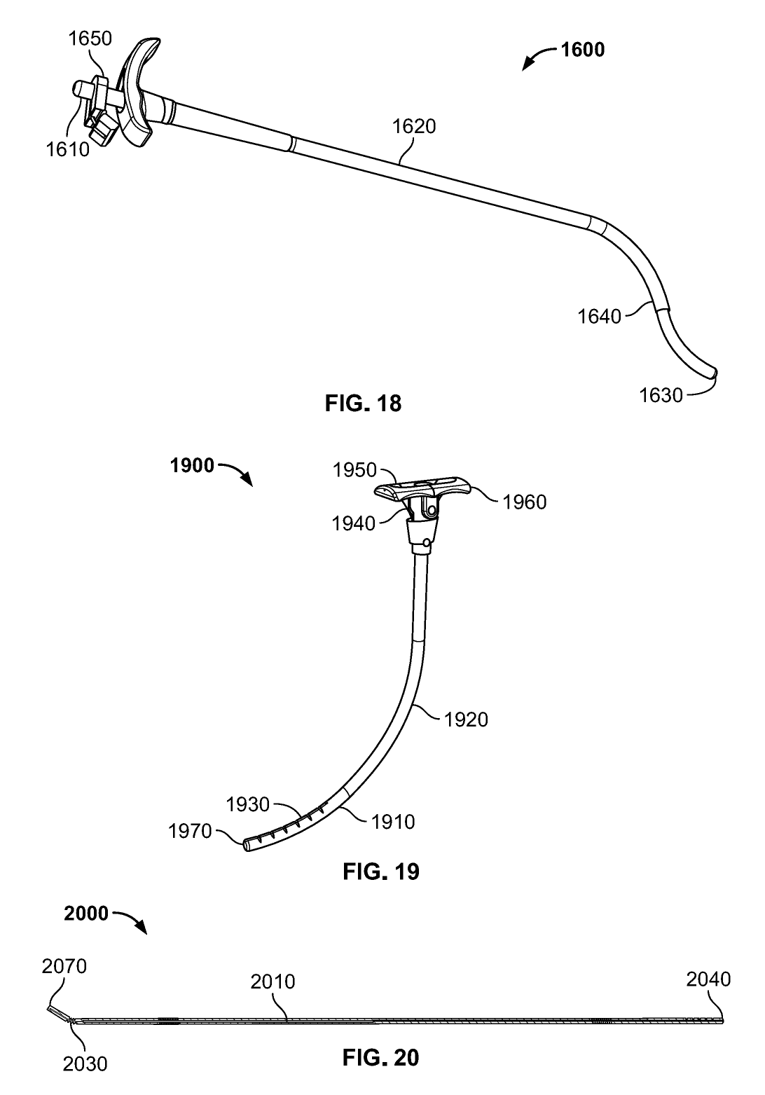

Moving now to FIG. 16, FIG. 17, and FIG. 18, a variation of the preferred embodiment is shown. As shown in FIG. 16, the device 1600 may come pre-assembled with the bougie 1610 already inserted into the stylet 1620 such that the tip 1630 of the boogie 1610 can be seen just passing out of the tip 1640 of the stylet 1620. The tip 1630 of the bougie 1610 optionally may be ball shaped or of a diameter equal to or greater than the diameter of the tip 1640 of the stylet 1620 to make it difficult to completely remove the bougie 1610 from the stylet 1620. A second actuator 1650 is coupled to the proximal portion of the bougie 1610 such that applying pressure to the second actuator 1650 causes the tip 1630 of the bougie 1610 to extend further past the tip 1640 of the stylet 1620 as shown in FIG. 17 and further in FIG. 18.

Now referring to FIG. 19. Another embodiment of the stylet 1910 is shown. At the distal end of the stylet 1910 is a plurality of spaced apart, transverse slots 1930 so positioned that the midsection of each slot is disposed on only one side of the stylet's 1910 longitudinal centerline. In another embodiment, the midsection of each slot may be on both sides of the stylet 1910 as well thus allowing for the stylet 1910 to be flexed in more than one direction. A cable 1940 running the inner length of the stylet 1910 connects the distal end of the stylet 1910 to a hinged portion 1950 of the actuator 1960 such that applying pressure to the hinged portion 1950 causes shortening of the cable 1940 resulting in flexion of the tip 1970 of the stylet 1910 in the direction the slots 1930 are disposed on the stylet 1910.

FIG. 20 further shows the inside of the bougie 2000. The bougie 2000 has a center area 2010. The center area 2010 houses a cable 2040 that runs the length of the inside of the bougie 2000. The cable 2040 connects the tip 2060 of the bougie 2000 with an actuator 2060. Such that when the actuator 2060 has pressured applied, the shortening of the cable 2040 occurs which results in the tip 2070 of the bougie 2000 to flex in the direction of the slots 2030 are disposed on the bougie 2000.

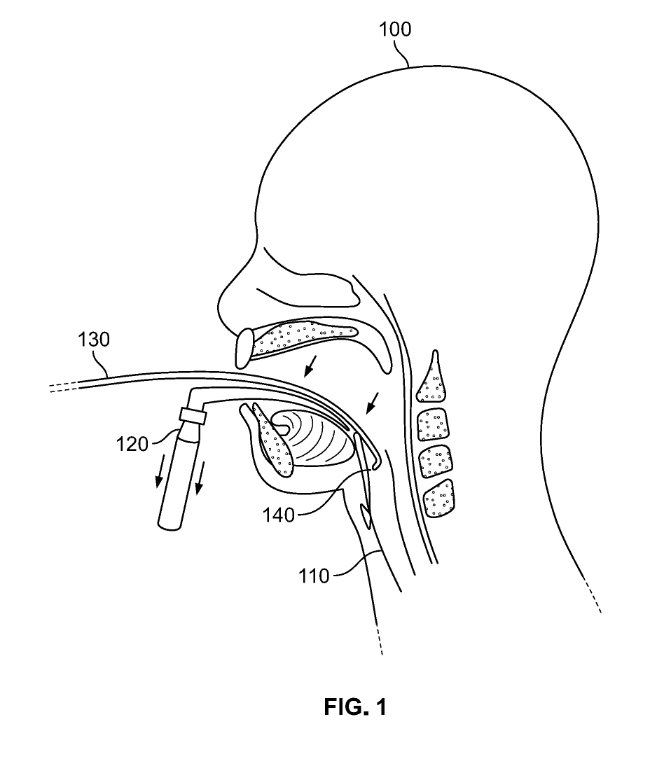

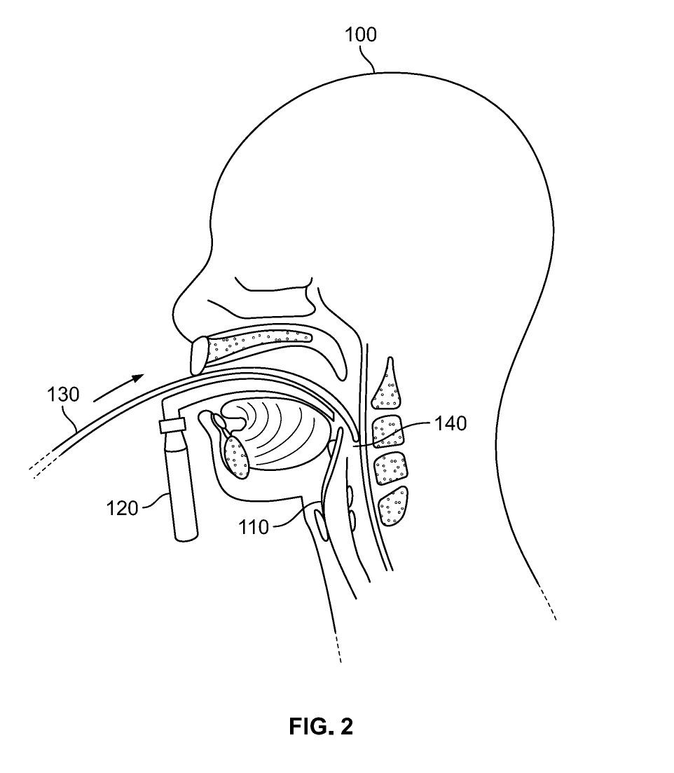

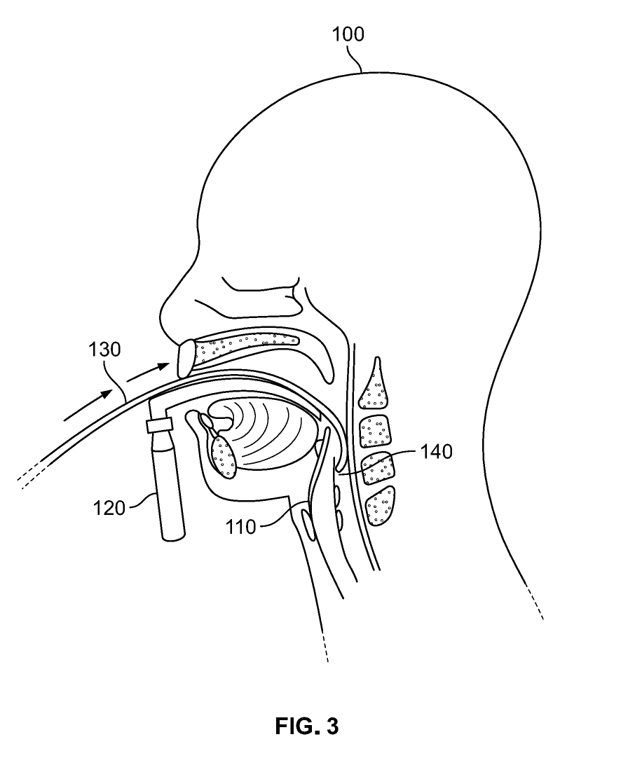

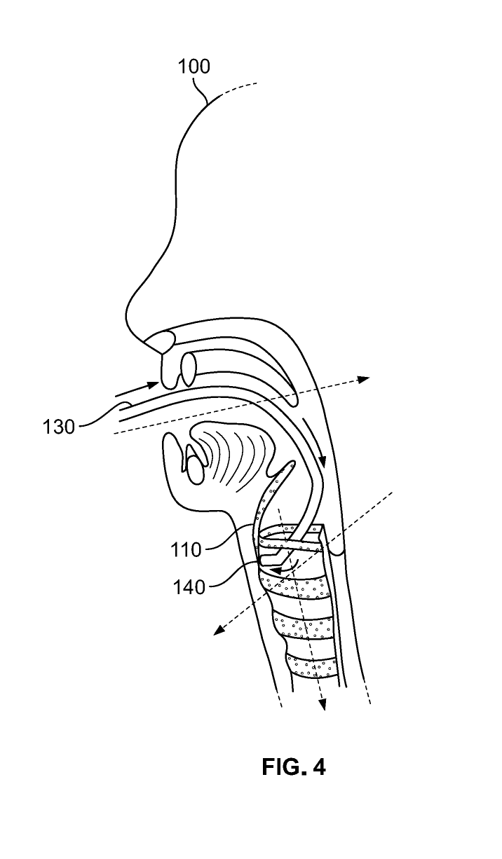

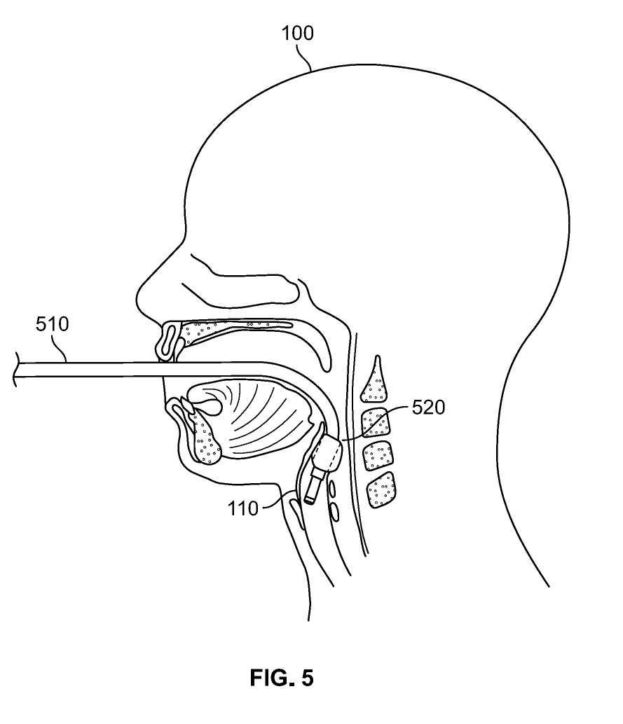

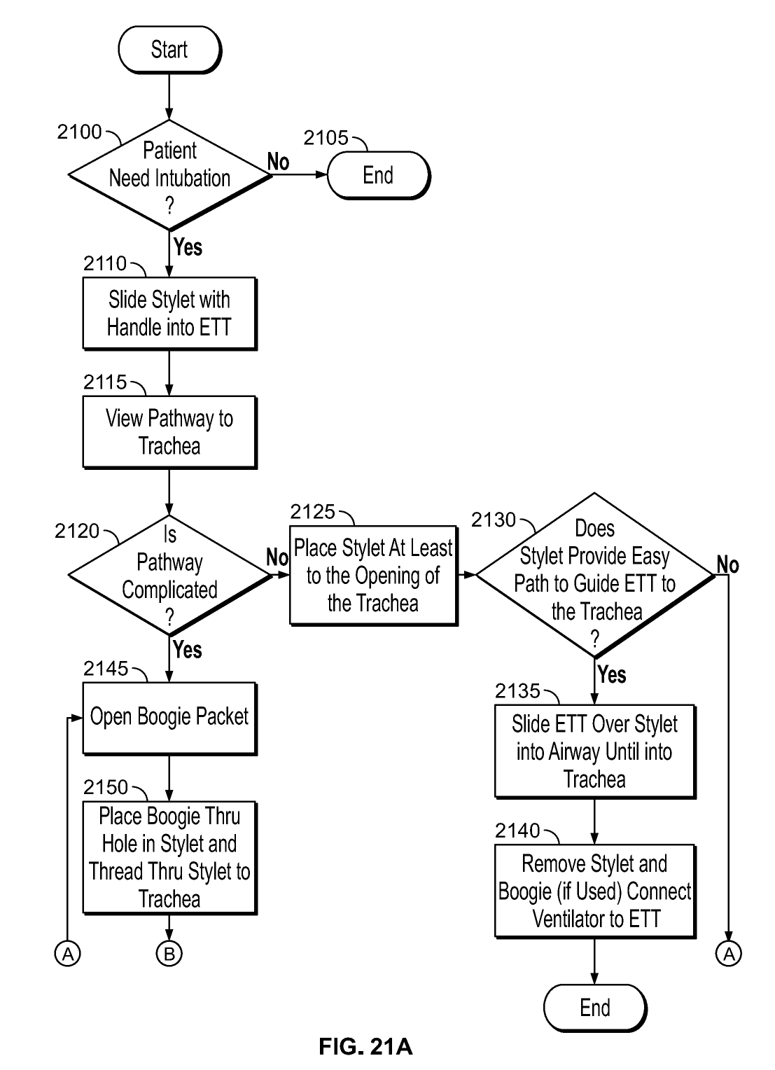

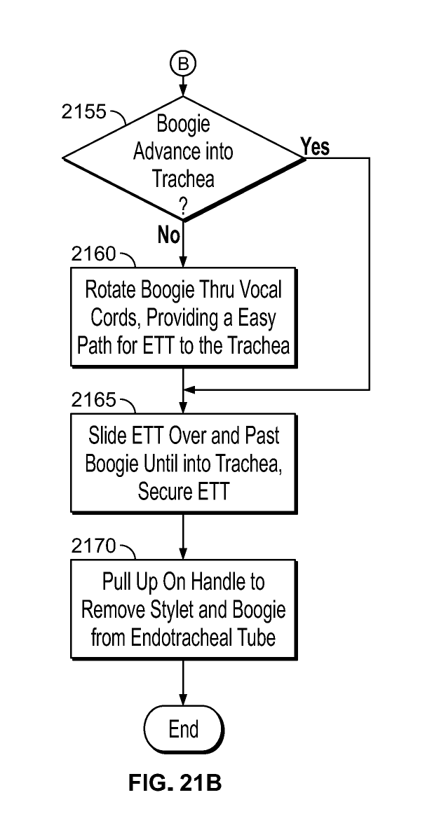

Referring now to FIG. 21A and FIG. 21B, references will be made to the drawings as the FIG. 1, FIG. 5, FIG. 21A and FIG. 21B as well, are described. A patient 100 is being prepared for a procedure of some type, the procedure can be a voluntary or emergency procedure in a medical facility or a procedure performed outside of the medical facility.

The medical provider determines if the patient 100 requires intubation 2100. If no intubation is required 2100, then no further activity in FIG. 21A and FIG. 21B are necessary and the process is exited 2105.

If the medical provider determines that the patient 100 requires intubation 2100, then a stylet 600 is slid into the ETT 510 at 2110. The stylet 600 has an actuator 620 at the proximal end 710 the stylet 600. The actuator 620 may be a handle, a knob, a dial, etc.

At 2115, the medical provider views the pathway to allow the stylet 600 to be followed such that the ETT 510 can be placed into the trachea of the patient. The medical provider determines if the pathway for the stylet 600 is complicated at 2120 and if it is determined to not be complicated, the medical provider positions and maneuvers the stylet 600 thru the vocal cords 110 of the patient 100 and near the opening to the trachea at 2125.

At 2130, if the medical practitioner is able to easily guide the ETT 510 into the trachea after placing the stylet 600, then, at 2135, the medical provider slides the ETT over the stylet 600 until the ETT 510 has passed the vocal cords and is placed in the trachea. The stylet 600 may be outside the trachea at this point or the tip 660 of the stylet 600 has entered the trachea. Once the ETT 510 has been placed in the trachea, at 2140, the medical provider utilizes the actuator 620 of the stylet 600 to remove the stylet 600 from the ETT 510 and the medical provider inflates the balloon 520 of the ETT 510 to secure the ETT 510 in the trachea of the patient 100. The ETT 510 is connected to the ventilator and the patient 100 is now intubated. The stylet 600 may be discarded by the medical provider at this point.

At 2130, after the medical provider has placed the stylet into the airway of the patient 100 the medical provider determines if the path does not provide an easy path for the ETT 510 to be placed into the trachea or at 2120 had determined that the pathway is complicated, then the medical provider removes the bougie 900 from its packet at 2145.

At 2150, the medical provider places the bougie 900 thru the opening 640 in the actuator 620 of the stylet 600.

At 2155, if the bougie has not been detected to have advanced into the trachea, then at 2160 the medical provider then threads the bougie 900 thru the stylet 600 applying pressure and at 2160 manipulates the proximal portion 910 of the bougie 900, which in turns allows the pattern 930 on the distal portion 920 of the bougie 900 to move the curved portion 940 and the tip 950 of the bougie to rotate thru the vocal cords and along the contour of the airway passage to enter the trachea and allow for an easy pathway for the ETT 510 to be placed in the trachea.

At 2155, if the tip 950 of the bougie 900 has progressed into the trachea of the patient 100, then proceed to the 2165.

At 2165, once the bougie has entered the trachea of the patient 100, then the medical practitioner slides the ETT 510 over and past the bougie 900 into the trachea. At 2170 the stylet 600 and bougie 900 are removed from the ETT 510 by pulling upon the actuator 620 of the stylet 600. The medical practitioner then connects the ventilator to the ETT 510 and discards the stylet 600 and bougie 900. The patient 100 is intubated at this point. The medical practitioner then inflates the balloon 520 of the ETT 510 to secure the ETT 510 in the trachea.

Other advantages of the stylet with an actuator is the ability for a medical provider to control and remove the stylet without having to discard the stylet on the floor, patient or other places. Thus, it is suitable for single patient use only and fills the need for affordable devices which can perform the function of much more costly devices without compromising the quality of patient care. Further the design and functionality of the bougie with at least one pattern allows for the bougie to be used when there are complicated or other conditions which cause for a complicated intubation, further the opening in the actuator allows for a faster intubation when there are complications arising from the inability of proper placement of the stylet and the ETT.

The features described with respect to one embodiment may be applied to other embodiments, or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A stylet for providing access to an airway of a patient for insertion of an endotracheal tube for intubation, the stylet (600) comprising:
a hollow tube, the hollow tube having a proximal end and a distal end;
the hollow tube having a shape determined prior to the hollow tube being placed at the airway of patient, the tube being pre-formed to be consistent with the entry point to the airway; and
an actuator coupled to the proximal end of the hollow tube enabling the release of the hollow tube from the endotracheal tube, wherein when the actuator (620) moves the hollow tube in an upward or downward position.

2. The stylet of claim 1 adapted for a single use.

3. The stylet of claim 1, further comprising a digital camera and a light, wherein the digital camera and the light are mounted at the distal end of the hollow tube enabling viewing of the airway of the patient and wherein the camera is in communication with a screen which enables viewing of the airway during intubation.

4. The stylet of claim 1, wherein the hollow tube is medical grade metal.

5. The stylet of claim 1, wherein the actuator is disposable medical grade plastic material.

6. The stylet of claim 1, wherein the actuator is a handle.

7. A method of intubating a patient, the method comprising:
an endotracheal tube mounted on a stylet;
inserting the stylet and the endotracheal tube into the trachea of a patient;
inserting the endotracheal tube past the distal end of the stylet into an airway of the patient, where the endotracheal tube extends past the stylet;
actuating an actuator coupled to the stylet to remove the stylet from the endotracheal tube; and
leaving the endotracheal tube in place, thereby intubating the patient.

8. The method of claim 7, further comprising:
disposing of the stylet and the actuator after a single use.

9. The method of claim 7, wherein the actuator is a handle.

10. The method of claim 7, further comprising:
when inserting the stylet to the trachea, the trachea not being reached by the stylet, inserting a bougie into an opening in the actuator coupled to the stylet, the bougie entering a proximal end of the stylet and extending past a distal end of the stylet, thereby entering the trachea and allowing for a path for the endotracheal tube to enter the trachea.

11. A bougie providing access to an airway of a patient for an endotracheal tube for intubation, the bougie comprising:
a rod, the rod having proximal end and a distal end;
at least a portion of the rod having one or more slots, at least one of the one or more slots having a pattern, where the pattern allows for the movement of the rod to be concentrated at the distal end when torque is applied at the proximal end of the rod to follow the contour of the airway;
the rod having a tip at the distal end and a curvature before the tip; and
the rod having the at least one slot between the proximal end and the curvature of the rod.

12. The bougie of claim 11 where the pattern of the one or more slots is a corkscrew.

13. The bougie of claim 11 where the tip of the rod is a coude tip.

14. The bougie of claim 11 where the tip is flexible for changing the angle.

15. The bougie of claim 11 adapted for a single use.

16. The bougie of claim 11, further comprising a digital camera and a light, wherein the digital camera and the light are mounted substantially at the distal end of the rod enabling viewing of the airway of the patient and wherein the camera is in communication with a screen which enables viewing of the airway during intubation.

17. The bougie of claim 11, wherein the rod is made of a disposable plastic material.

18. The bougie of claim 11, wherein at least one of the one or more portions of the rod is smooth.
